(19) United States
(12) Patent Application Publication
(10) Pub. No.: US 2017/0251964 A1
(43) Pub. Date: Sep. 7, 2017

(54) BLOOD SAMPLING DEVICE

(71) Applicant: MEDIFUN CORPORATION, Taichung City (TW)

(72) Inventors: William Chen, Taichung City (TW); HAO-CHENG FAN, Taichung City (TW)

(21) Appl. No.: 15/062,182

(22) Filed: Mar. 6, 2016

(51) Int. Cl. A61B 5/15; A61B 5/151; A61B 17/3209

(52) U.S. Cl. CPC A61B 5/150022; A61B 17/32093; A61B 5/15142; A61B 5/1513; A61B 5/15144

(57) ABSTRACT

A blood sampling device contains: a body, a fitting sleeve, a triggering block, a trigger spring, and a switch. The body is hollow and fits with the fitting sleeve which includes an exit for extending a lancet out of the exit, and the body includes a first fixing position and a second fixing position. The first fixing position is an untriggering position away from the exit, and the second fixing position is a triggering position close to the exit. A first end of the triggering block abuts against the trigger spring, the triggering block includes a connecting orifice for accommodating the lancet and includes an elastic piece retaining at the first fixing position or the second fixing position. The switch is mounted on the body and includes a releasing segment corresponding to the first fixing position so as to release the elastic piece from the first fixing position.

### FIELD OF THE INVENTION

The present invention relates to a blood sampling device which is capable of replacing and positioning a new lancet in an untriggering position quickly.

### BACKGROUND OF THE INVENTION

A conventional blood sampling device is employed to sample patient's bloods after inserting its lancet into patient's skin.

Another conventional blood sampling device is disclosed in TW Patent No. M482372 and contains a body, a triggering block, a pull member, a movable seat, a fitting sleeve, and a lancet. The lancet is inserted onto the movable seat, and the pull member is pulled backward so as to drive the movable seat to move toward an untriggering position, hence a switch is pressed to trigger the lancet to pierce into the patient's skin, and patient's bloods are sampled. Preferably, the fitting sleeve is adjusted to control a depth of the lancet into the patient's skin.

However, in operation, the lancet is inserted into the patient's skin, the pull member is pulled to drive the lancet to move toward the untriggering position, and the switch is pressed to trigger the lancet, thus causing troublesome blood sampling.

The present invention has arisen to mitigate and/or obviate the afore-described disadvantages.

### SUMMARY OF THE INVENTION

The primary objective of the present invention is to provide a blood sampling device which is capable of replacing and positioning a new lancet in an untriggering position quickly.

To obtain the above objective, a blood sampling device provided by the present invention contains: a body, a fitting sleeve, a triggering block, a trigger spring, and a switch.

The body is a hollow tube and is fitted with the fitting sleeve, the fitting sleeve includes an exit defined on one end thereof so as to extend a lancet out of the exit, and the body includes a first fixing position and a second fixing position which are formed in the body.

The first fixing position is a untriggering position away from the exit, and the second fixing position is a triggering position close to the exit, the triggering block is movably disposed in the body and its first end abuts against the trigger spring, the triggering block includes a connecting orifice defined on a second end thereof and corresponding to the exit so as to accommodate the lancet, the triggering block also includes an elastic piece arranged on an outer wall thereof and alternatively retaining at the first fixing position or the second fixing position, the switch is mounted on an outer wall of the body and includes a releasing segment inserting into the body and corresponding to the first fixing position so as to release the elastic piece from the first fixing position.

Preferably, the body further includes a guiding rail on which the switch slides back and forth, the triggering block further includes a receiving groove defined therein and communicating with the connecting orifice, and the switch includes a pushing segment extending into the receiving groove.

Preferably, the body further includes a partition arranged therein, and a first side of the partition abuts against the trigger spring, the body further includes a returning spring abutting against a second side of the partition, the triggering block further includes a pull bar extending through the trigger spring, the partition, and the returning spring and retaining with the returning spring.

Preferably, the body includes a sheath fitted thereon, and the first fixing position and the second fixing position are formed in the sheath, wherein the sheath has at least one guiding track arranged on an inner wall thereof, and the triggering block is housed in the sheath and includes at least one rib formed on the outer wall thereof and corresponding to the at least one guiding track, respectively.

Preferably, the triggering block further includes a flexible trench defined on the outer wall thereof and communicating with the connecting orifice.

### BRIEF DESCRIPTION OF THE DRAWINGS

FIG. 1 is a perspective view showing the exploded components of a blood sampling device according to a preferred embodiment of the present invention.

FIG. 2 is a perspective view showing the assembly of the blood sampling device according to the preferred embodiment of the present invention.

FIG. 3 is a cross sectional view showing the assembly of the blood sampling device according to the preferred embodiment of the present invention.

FIG. 4 is a cross sectional view showing the operation of the blood sampling device according to the preferred embodiment of the present invention.

FIG. 5 is another cross sectional view showing the operation of the blood sampling device according to the preferred embodiment of the present invention.

FIG. 6 is a cross sectional view of a portion A of FIG. 5.

FIG. 7 is also another cross sectional view showing the operation of the blood sampling device according to the preferred embodiment of the present invention.

FIG. 8 is a cross sectional view of a portion B of FIG. 7.

FIG. 9 is still another cross sectional view showing the operation of the blood sampling device according to the preferred embodiment of the present invention.

FIG. 10 is another cross sectional view showing the operation of the blood sampling device according to the preferred embodiment of the present invention.

FIG. 11 is also another cross sectional view showing the operation of the blood sampling device according to the preferred embodiment of the present invention.

### DETAILED DESCRIPTION OF THE PREFERRED EMBODIMENTS

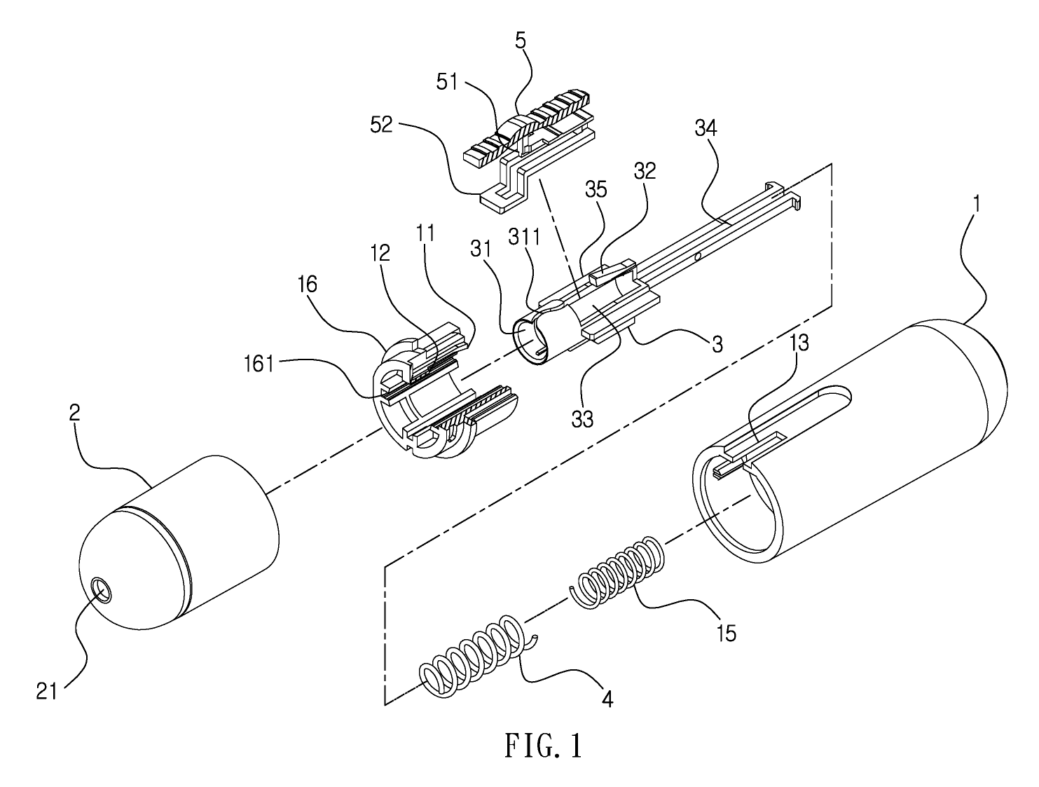

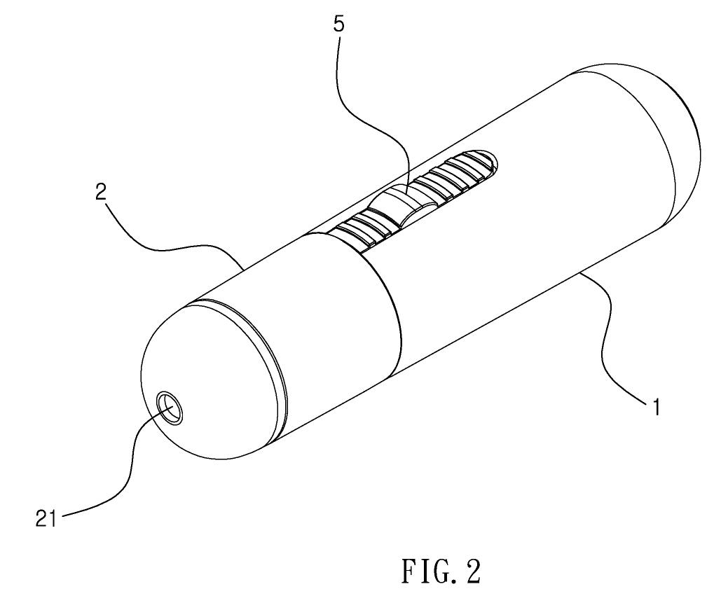

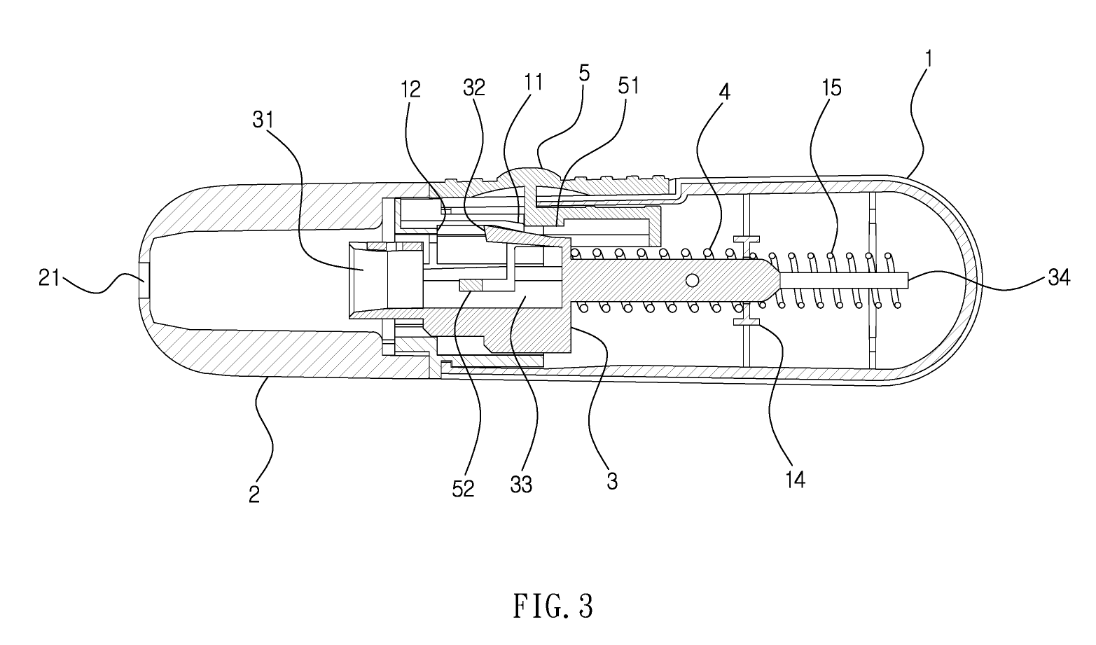

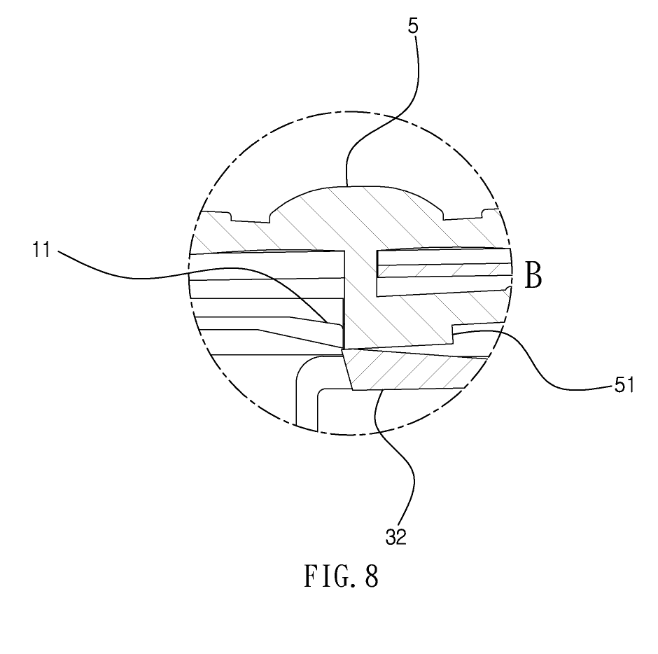

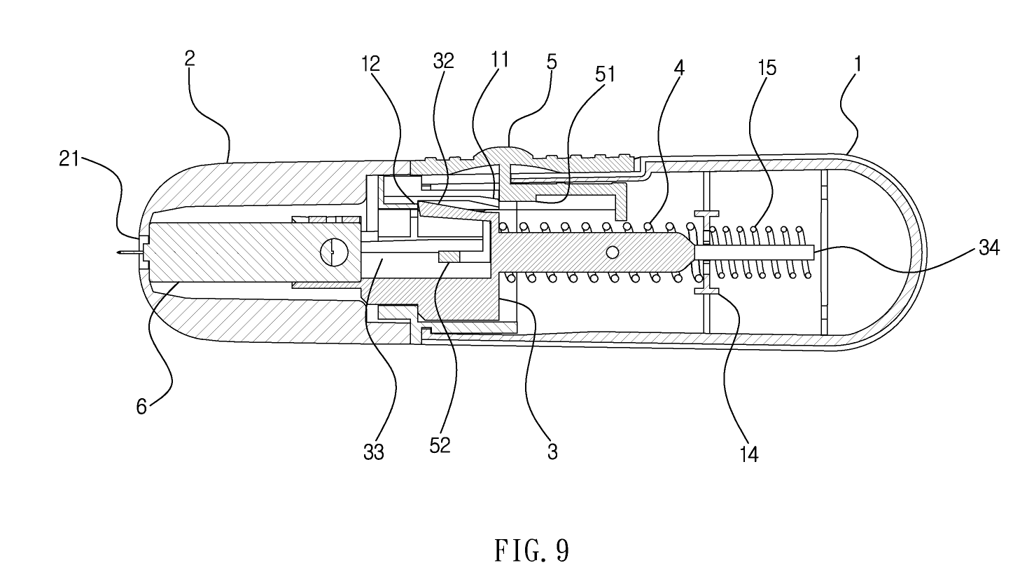

With reference to FIGS. 1 to 11, a blood sampling device according to a preferred embodiment of the present invention is employed to test blood sugar and is capable of replacing and positioning a new lancet in a untriggering position quickly, the blood sampling device comprises: a body 1, a fitting sleeve 2, a triggering block 3, a trigger spring 4, and a switch 5.

The body 1 is a hollow tube and is fitted with the fitting sleeve 2. The fitting sleeve 2 includes an exit 21 defined on one end thereof so as to extend the lancet 6 out of the exit 21, and the body 1 includes a first fixing position 11 and a second fixing position 12 which are formed in the body 1, wherein the first fixing position 11 is the untriggering position away from the exit 21, and the second fixing position 12 is a triggering position close to the exit 21. The triggering block 3 is movably disposed in the body 1 and its first end abuts against the trigger spring 4, the triggering block 3 includes a connecting orifice 31 defined on a second end thereof and corresponding to the exit 21 so as to accommodate the lancet 6. The triggering block 3 also includes an elastic piece 32 arranged on an outer wall thereof and alternatively retaining at the first fixing position 11 or the second fixing position 12. The switch 5 is mounted on an outer wall of the body 1 and includes a releasing segment 51 inserting into the body 1 and corresponding to the first fixing position 11 so as to release the elastic piece 32 from the first fixing position 11.

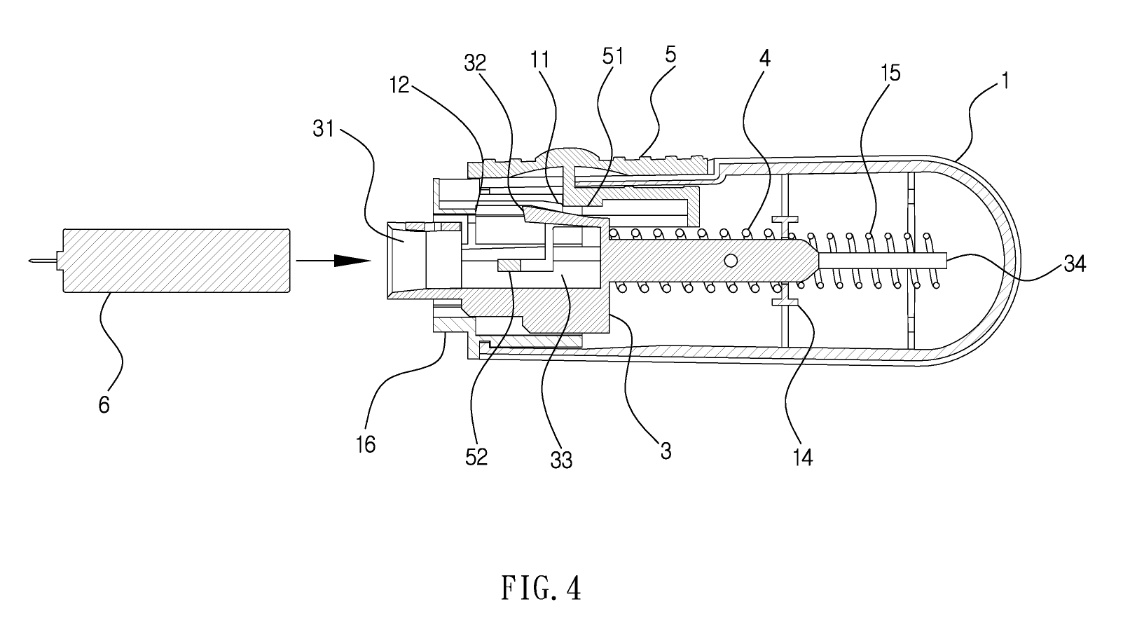

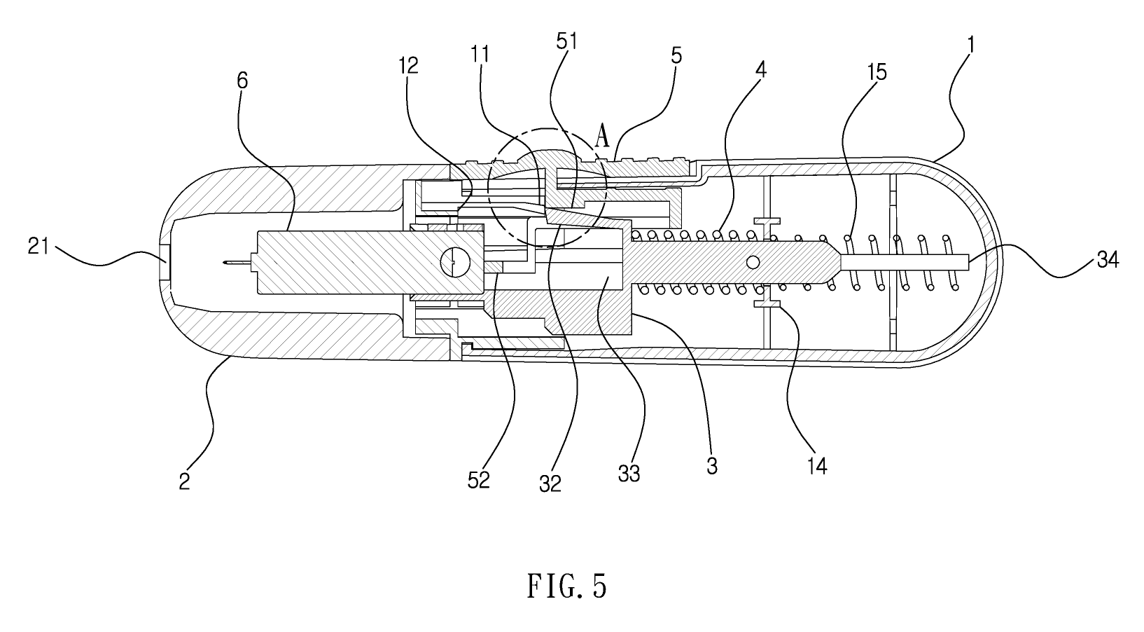

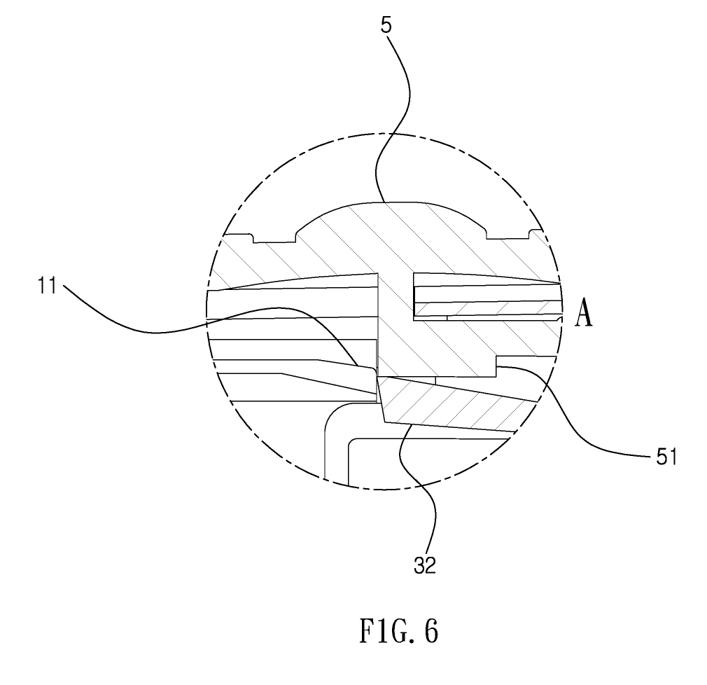

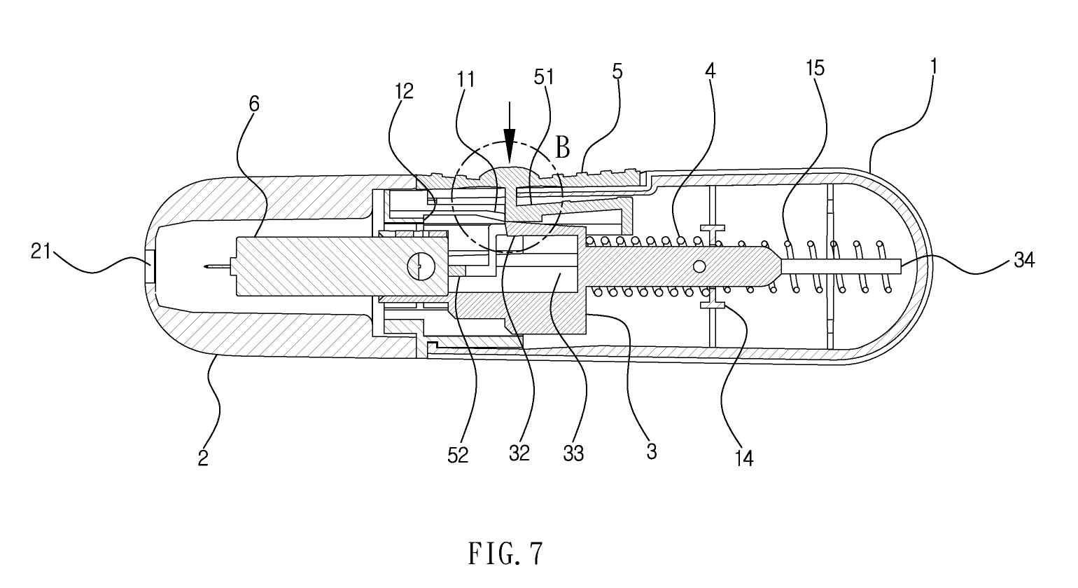

After removing the fitting sleeve 2 from the body 1, the lancet 6 is accommodated in the connecting orifice 31 of the triggering block 3, and the triggering block 3 is pushed to move inwardly and to press the trigger spring 4 (as shown in FIG. 4). Thereafter, the elastic piece 32 corresponds to and retains at the first fixing position 11, hence the lancet 6 is limited at the untriggering position (as illustrated in FIGS. 5 and 6), and the fitting sleeve 2 is fitted with the body 1. After pressing the switch 5, the releasing segment 51 pushes the elastic piece 32 to remove from the first fixing position 11, and the triggering block 3 is urged by the trigger spring 4 to push the lancet 6 out of the exit 21 (as shown in FIGS. 7 to 9).

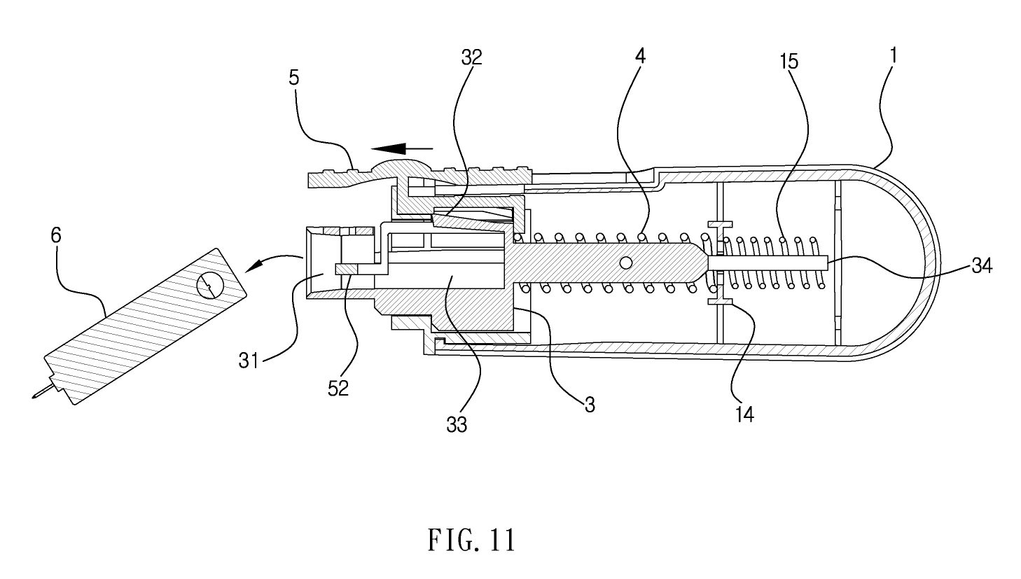

The body 1 further includes a guiding rail 13 on which the switch 5 slides back and forth. The triggering block 3 further includes a receiving groove 33 defined therein and communicating with the connecting orifice 31, and the switch 5 includes a pushing segment 52 extending into the receiving groove 22, such that after the lancet 6 samples bloods, the fitting sleeve 2 is removed by user and the switch 5 is pushed so that the pushing segment 52 pushes the lancet 6 out of the connecting orifice 31, thus replacing the lancet 6 (as illustrated in FIG. 11).

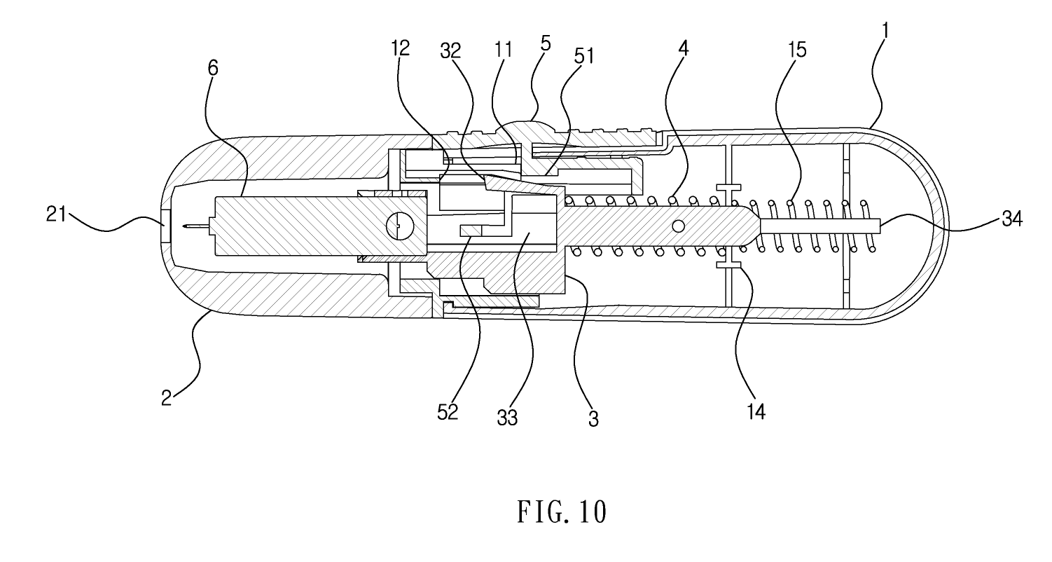

The body 11 further includes a partition 14 arranged therein, and a first side of the partition 14 abuts against the trigger spring 4, the body 11 further includes a returning spring 15 abutting against a second side of the partition 14. The triggering block 3 further includes a pull bar 34 extending through the trigger spring 4, the partition 14, and the returning spring 15 and retaining with the returning spring 15, such that when the lancet 6 pierces into patient's skin to sample the bloods, the returning spring 15 pulls the triggering block 3 back to an original position, thus retracting the lancet 6 quickly (as shown in FIG. 10).

Furthermore, the body 1 includes a sheath 16 fitted thereon, and the first fixing position 11 and the second fixing position 12 are formed in the sheath 16, wherein the sheath 16 has at least one guiding track 161 arranged on an inner wall thereof, the triggering block 3 is housed in the sheath 16 and includes at least one rib 35 formed on the outer wall thereof and corresponding to the at least one guiding track 161, respectively, such that the triggering block 3 slides in the body 1 smoothly by way of the at least one guiding track 161 and the at least one rib 25.

The triggering block 3 further includes a flexible trench 311 defined on the outer wall thereof and communicating with the connecting orifice 31, hence the connecting orifice 31 deforms by using the flexible trench 311.

Preferably, the blood sampling device of the present invention is capable of positioning the new lancet at the untriggering position quickly, after replacing the lancet.

While the preferred embodiments of the invention have been set forth for the purpose of disclosure, modifications of the disclosed embodiments of the invention and other embodiments thereof may occur to those skilled in the art. Accordingly, the appended claims are intended to cover all embodiments which do not depart from the spirit and scope of the invention.

What is claimed is:

1. A blood sampling device comprising: a body, a fitting sleeve, a triggering block, a trigger spring, and a switch;
the body being a hollow tube and being fitted with the fitting sleeve, the fitting sleeve including an exit defined on one end thereof so as to extend a lancet out of the exit, and the body including a first fixing position and a second fixing position which are formed in the body, wherein the first fixing position is a untriggering position away from the exit, and the second fixing position is a triggering position close to the exit, the triggering block is movably disposed in the body and its first end abuts against the trigger spring, the triggering block includes a connecting orifice defined on a second end thereof and corresponding to the exit so as to accommodate the lancet, the triggering block also includes an elastic piece arranged on an outer wall thereof and alternatively retaining at the first fixing position or the second fixing position, the switch is mounted on an outer wall of the body and includes a releasing segment inserting into the body and corresponding to the first fixing position so as to release the elastic piece from the first fixing position.

2. The blood sampling device as claimed in claim 1, wherein the body further includes a guiding rail on which the switch slides back and forth, the triggering block further includes a receiving groove defined therein and communicating with the connecting orifice, and the switch includes a pushing segment extending into the receiving groove.

3. The blood sampling device as claimed in claim 1, wherein the body further includes a partition arranged therein, and a first side of the partition abuts against the trigger spring, the body further includes a returning spring abutting against a second side of the partition, the triggering block further includes a pull bar extending through the trigger spring, the partition, and the returning spring and retaining with the returning spring.

4. The blood sampling device as claimed in claim 1, wherein the body includes a sheath fitted thereon, and the first fixing position and the second fixing position are formed in the sheath, wherein the sheath has at least one guiding track arranged on an inner wall thereof, and the triggering block is housed in the sheath and includes at least one rib formed on the outer wall thereof and corresponding to the at least one guiding track, respectively.

5. The blood sampling device as claimed in claim 1, wherein the triggering block further includes a flexible trench defined on the outer wall thereof and communicating with the connecting orifice.
